(19) United States
(12) Patent Application Publication
(10) Pub. No.: US 2017/0258388 A1
(43) Pub. Date: Sep. 14, 2017

(54) DEVICES AND METHODS FOR MEASURING BALANCE

(71) Applicants: Ana Cecilia Gomez del Campo, Atlanta, GA (US); Franklin Hailey Brown, Atlanta, GA (US); Matthew Devlin, Atlanta, GA (US); Garrett Wallace, Atlanta, GA (US)

(72) Inventors: Ana Cecilia Gomez del Campo, Atlanta, GA (US); Franklin Hailey Brown, Atlanta, GA (US); Matthew Devlin, Atlanta, GA (US); Garrett Wallace, Atlanta, GA (US)

(21) Appl. No.: 15/451,763

(22) Filed: Mar. 7, 2017

Related U.S. Application Data
(60) Provisional application No. 62/305,087, filed on Mar. 8, 2016.

(51) Int. Cl. A61B 5/00; A61B 5/103

(52) U.S. Cl. CPC A61B 5/4023; A61B 5/6887; A61B 5/1036; A61B 2562/0247

(57) ABSTRACT

Devices and methods for measuring balance in a subject are described herein. The devices include a platform, a mechanism connected to the platform, and at least one sensor in or on the platform. The at least one sensor measures the balance of a subject atop the platform as the mechanism induces a platform movement in the horizontal plane. A data set measured by the at least one sensor can processed and then used to assess a subject's balance.

### CROSS-REFERENCE TO RELATED APPLICATIONS

This application, filed 7 Mar. 2017, under 35 U.S.C. §119(e), claims the benefit of U.S. Provisional Patent Application Ser. No. 62/305,087, filed 8 Mar. 2016, entitled “WOBBLE,” the entire contents and substance of which are hereby incorporated by reference as if fully set forth below.

### TECHNICAL FIELD

The various embodiments of the disclosure relate generally to processes, methods, and systems for measuring and assessing balance in a subject. It is particularly useful for monitoring and diagnosing neurological issues which can arise through brain trauma such as concussion or through chemical impairment such as drug use. Further applications can include assessing orthopedic injuries and balance training such as physical or occupational therapy.

### BACKGROUND

Concussions are a serious public health problem. There are 300,000 sport-related concussions every year and 23% of deployed military service members sustain concussions. According to the CDC, mild traumatic brain injuries cost the US economy close to $22 billion annually. If a patient returns to activity before making a full recovery, they are at an increased risk of sustaining multiple concussions and even a life-threatening second impact. Because concussion inhibits sensory integration, the robustness of the balance control system is one biomarker for concussion recovery.

Balance tests can also provide a corollary for brain health and overall awareness. For example, the field sobriety test remains prevalent in assessing driver impairment even after the invention of the breathalyzer by Robert Borkenstein. The breathalyzer allowed police officers to get immediate, accurate biological information to determine if someone was not physically fit to operate a vehicle. Psychoactive drugs (stimulants, sedatives, antidepressants, marijuana, and narcotic analgesics) pose a similar public health concern when they are abused or misused by drivers. According to the 2014 National Survey on Drug Use and Health (NSDUH) 10 million people reported driving under the influence of illicit drugs in the past year. However, a method for detecting drug-impaired driving with the same speed and accuracy as a breathalyzer has yet to be discovered.

### BRIEF SUMMARY

The various embodiments of the disclosure relate generally to processes, methods, and systems for balance assessment, training and characterization. These embodiments aim to provide a cost-effective, objective, and easy-to-use solution for clinicians, therapists, and others who encounter balance deficiencies.

An embodiment of the disclosure can include a device for measuring balance in a subject positioned thereon. The device can include: a platform, a sensor, and a mechanism connected to the platform. The mechanism can induce a platform movement along a horizontal plane and the sensor can measure a data set containing the subject's response to the platform movement.

The device for measuring balance can induce a platform movement which comprises an x-direction and a y-direction, independent of one another and optionally oriented perpendicular to one another in the horizontal plane. The platform movement may also include a platform displacement, a platform velocity, and platform acceleration. Additionally, the platform displacement, the platform velocity, and the platform acceleration may each comprise an x-direction, a y-direction or a combination of both.

Embodiments of the disclosure can also include a platform reset after the platform movement. The platform reset may comprise a reset time and a neutral platform position.

In embodiments of the device, the sensor can include a load sensor or a pressure sensor for measuring force of the subject positioned on the platform. Further some devices may include 1 sensor, 2 sensors, 3 sensors or at least 4 sensors. In any of the previous embodiments, the sensor can be integrated in or on the platform. Additionally in any of the previous embodiments, the sensor can be positioned at or proximate the perimeter of the platform.

An embodiment of the disclosure can further include a base. Preferably, the base is connected to the mechanism at a position different from the platform.

Embodiments of the disclosure can further include a storage location. The storage location may provide a location to store and/or access the data set measured by the sensor.

Embodiments of the disclosure may further include a data processor and an interface. The data processor can be configured to generate a real-time analysis of the data set and the interface can display the real-time analysis.

An embodiment of the disclosure can include a device for measuring balance containing a mechanism that includes 1 actuator, 2 actuators, or at least 4 actuators.

Embodiments of the disclosure can also include an adapter configured to connect a power source to the device.

An embodiment of the disclosure can include a device for measuring balance that includes a substantially rectangular platform, a mechanism connected to the platform and 4 load cells positioned proximate to the platform perimeter. The device can induce a platform movement comprising an x-direction and a y-direction, independent of one another and oriented perpendicular to one another in the horizontal plane. Alternatively, the device can induce a platform movement comprising an x-direction displacement, a y-direction displacement, a platform velocity, and a platform acceleration. In another embodiment, the device can further induce a platform reset after the platform movement, wherein the platform reset comprises a reset time and a neutral platform position.

Devices for measuring balance can further include electronic or mechanical controls to limit operations such as the platform movement. In an embodiment, the device can induce a platform movement that comprises a platform acceleration of about 0.6 g or less.

An embodiment of the disclosure can include a method for measuring balance in a subject including: positioning the subject on a platform, wherein the platform comprises at least one sensor and is configured to move along a horizontal plane; inducing a series of platform movements in the horizontal plane; and measuring a data set comprising the subject's response to each platform movement using at least one sensor incorporated in or on the platform.

Methods of the disclosure can further include a platform reset after each platform movement, wherein the platform reset comprises a reset time and a neutral platform position.

A method of the disclosure can further include saving the data set in a storage location, wherein the storage location provides a reference tag comprising a subject ID and a session ID. Another method of the disclosure can further include processing the data using real-time analysis and displaying a balance result on an interface.

A method of the disclosure can include measuring a data set using a load sensor or a pressure sensor to measure force of the subject positioned on the platform. Methods of the disclosure may include a platform comprising 1 sensor, 2 sensors, 3 sensors, or at least 4 sensors. Methods of the disclosure may also include measuring a data set using a sensor positioned proximate the perimeter of the platform.

Methods of the disclosure can include inducing a series of platform movements using a mechanism comprising 1 actuator, 2 actuators, or at least 4 actuators. Additionally, embodiments of the disclosure can further comprise providing a power source to transfer energy to a mechanism connected to the platform.

Another embodiment of the disclosure can include a method for assessing balance in a subject including the steps: positioning the subject on a platform, wherein the platform comprises at least one sensor and is configured to move along a horizontal plane; inducing a series of platform movements in the horizontal plane; measuring a data set comprising the subject's response to each platform movement using at least one sensor incorporated in or on the platform; storing the data set in a storage location, wherein the storage location provides a reference tag associated with the data set comprising a subject ID and a session ID; and reporting a balance assessment for the subject.

Methods of assessing balance can include inducing a series of platform movements. In one embodiment, each platform movement may comprise an x-direction and a y-direction, independent of one another and oriented perpendicular to one another in the horizontal plane. In a second embodiment, each platform movement can include an x-direction displacement, a y-direction displacement, a platform velocity, and a platform acceleration. As described herein, each platform movement can be substantially random, such that one or more of parameters from the following list is determined at random: the x-direction displacement, the y-direction displacement, the platform velocity, and the platform acceleration. Alternatively methods of the disclosure can include inducing a series of platform movements wherein each platform movement is selected or set by a user. As described herein, each platform movement can be selected or set, such that one or more of parameters from the following list is predetermined or adjusted in real-time: the x-direction displacement, the y-direction displacement, the platform velocity, and the platform acceleration. Embodiments of the disclosure can also include methods of assessing balance that include inducing a series of platform movements, wherein one or more platform movement can be substantially random and one or more platform movement can be selected or set by a user. Therefore methods of use should not be held as constrained to a single platform movement when inducing a series of platform movements. Each platform movement is an independent event which can be modified or adjusted as provided by embodiments of the disclosure.

A method of the disclosure for assessing balance can include inducing a series of 5, 6, 7, 8, 9 or at least 10 platform movements. Each of the platform movements can be random or set and may further include a platform reset after the platform movement. Additionally, methods of assessing balance can further include controlling the difficulty of the series of platform movements such that the magnitude of the platform acceleration can be set to increase or remain the same after each platform movement.

Another embodiment of the disclosure can include inducing a series of at least 5 platform movements wherein the subject is positioned on one leg for at least 4 platform movements.

A method of the disclosure for assessing balance can include reporting the balance assessment comprising a diagnosis using characteristics including the data set. As described herein, reporting a diagnosis may include a condition selected from the group consisting of: concussion, inebriation, non-alcohol related drug impairment, central-nervous system depression, or combinations thereof.

Embodiments of the disclosure may further include tracking the subject balance by comparing at least two data sets, wherein the reference tag associated with each data set notes the same subject ID and notes different session IDs.

Embodiments of the present disclosure are not limited to balance tracking for concussions, as many neurological deficits can manifest as balance deficits. Further orthopedic injuries can require extensive physical therapy and training, which can be aided by a robust balance assessment methods and devices. The embodiments provided herein provide a cost effective and objective solution for assessing, monitoring, and training subject balance.

### BRIEF DESCRIPTION OF THE DRAWINGS

FIG. 1a illustrates a profile view of a device in accordance with an exemplary embodiment of the disclosure. FIG. 1b illustrates an elevated perspective view of a device in accordance with an exemplary embodiment of the disclosure.

FIG. 2 illustrates an exploded perspective view of a device in accordance with an exemplary embodiment of the disclosure.

FIG. 3 illustrates an exploded perspective view of a device in accordance with an exemplary embodiment of the disclosure.

FIG. 4 provides a photograph of a device in accordance with an exemplary embodiment of the disclosure.

FIG. 5 provides a photograph of a device, in accordance with an exemplary embodiment of the disclosure.

FIG. 6a provides a graph of a data set showing mean-referenced medial-lateral CoP as a function of time, and FIG. 6b provides a graph of a data set showing mean-referenced anterior-posterior CoP as a function of mean-referenced medial-lateral CoP, in accordance with exemplary embodiments of the disclosure.

FIG. 7 provides a flow chart showing the flow of information from the balance platform to a database, an interface, and a health record accordance with an exemplary embodiment of the disclosure.

### DETAILED DESCRIPTION

Although preferred embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. The disclosure is capable of other embodiments and of being practiced or carried out in various ways. Also, in describing the preferred embodiments, specific terminology will be resorted to for the sake of clarity.

It must also be noted that, as used in the specification and the appended claims, the singular forms “a,” “an” and “the” include plural referents unless the context clearly dictates otherwise.

Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Disclosed are various devices and methods for measuring balance in a subject positioned atop the device. Aspects of the present disclosure may be used to identify balance issues, assess the severity of balance issues, and/or train balance-related motor skills.

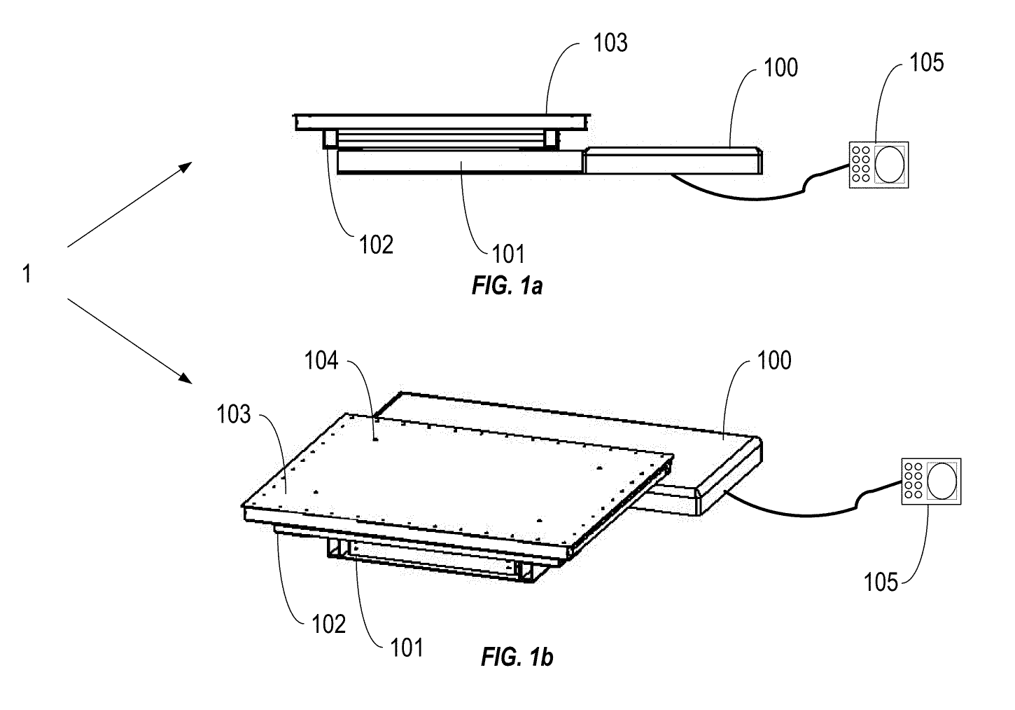

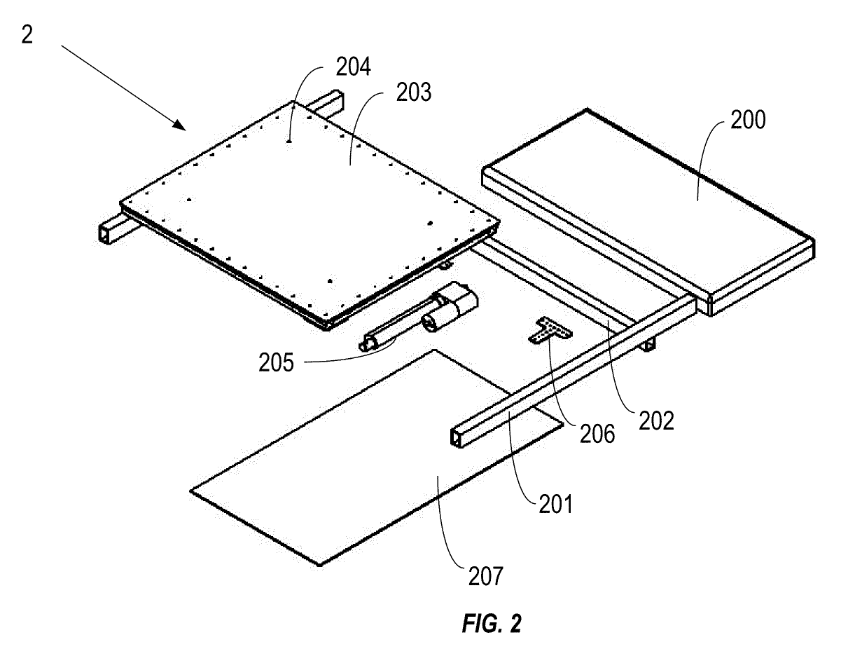

Devices for measuring balance in a subject positioned thereon may include a platform, a sensor, and a mechanism for inducing a platform movement. In a non-limiting, exemplary embodiment, FIGS. 1a and 1b demonstrate a device 1 for measuring balance as provided in embodiments of the disclosure. The device includes an x-direction support rail 101, a y-direction support rail 102, and platform 103. The device 1 also includes one or more sensors, for example at position 104, and an interface 105 connected to the device. Another non-limiting, exemplary embodiment is provided in FIG. 2, which shows a device 2. The device 2 includes a base 200, which may or may not include a beveled edge as shown; an x-direction support rail 201; a y-direction support rail 202, a platform 203 and one or more sensors, for example at position 204. The device for measuring balance 2 can also include exterior and interior components such as one or more mechanisms connected to platform 203; for example actuator 205 that can induce movement of platform 203, one or more braces such as 206, and an exterior housing 207.

In the devices described herein, the mechanism can include one or more components oriented in various directions. An exemplary device may include two components: a first component oriented in an x-direction and a second component oriented in the y-direction. The first component and second component can be oriented such that the x-direction and y-direction are perpendicular to one another in a horizontal plane normal to gravity. Alternatively, the components can be oriented in a non-perpendicular arrangement. Device components can act and operate independent of one another. For example, a device may include two components oriented in an x-direction and a y-direction, but only induce a platform movement in a either the x-direction or the y-direction. Alternatively, the two components could operate at an equivalent rate, inducing a movement that bisects the two directions of the components. Alternatively, the two components could operate at different rates. As shown in FIG. 2, a non-limiting, exemplary device 2 for measuring balance can include an x-direction support rail 201 and an actuator 205, which in combination can provide a mechanism for inducing a platform movement.

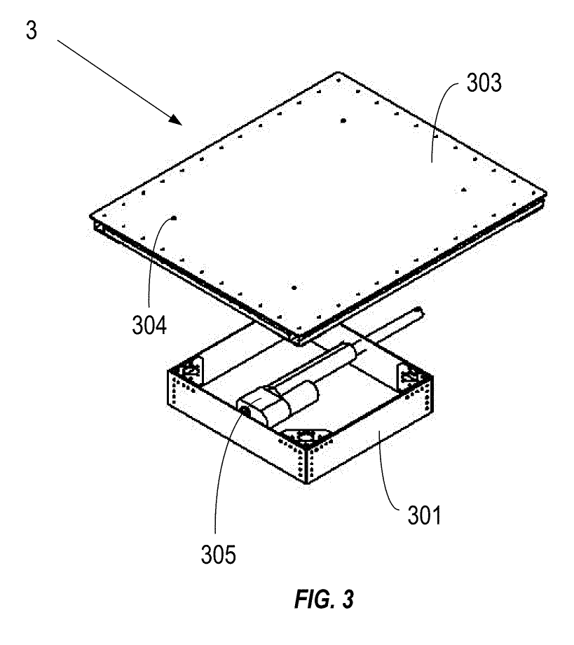

FIG. 3 shows another aspect of the disclosure. A non-limiting exemplary device 3 can include a structure 301 which can support the other components, a platform 303, one or more sensor places in the device, such as at place 304, and an actuator 305 attached to platform 303.

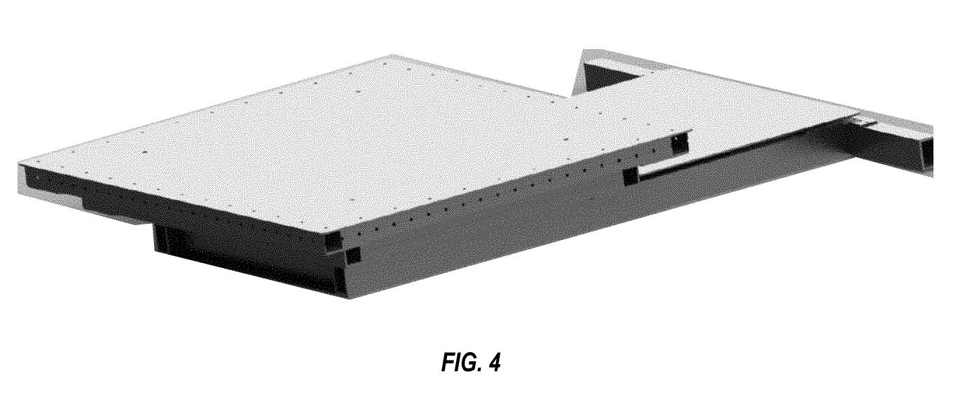

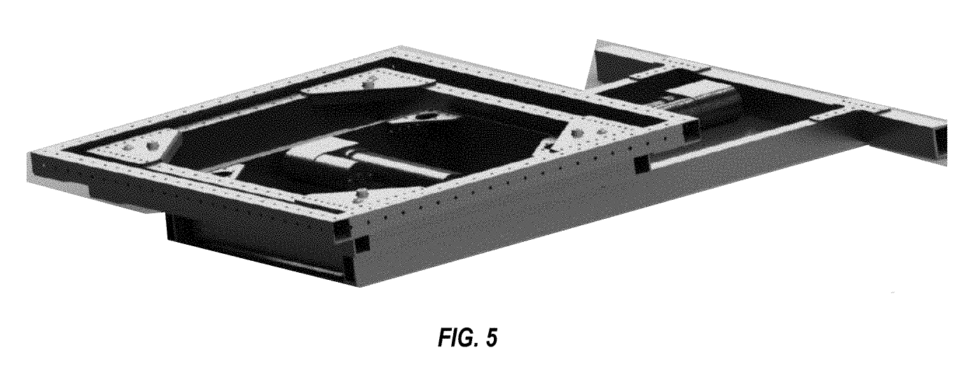

The elements of the disclosure, including a platform, a sensor, a mechanism for inducing movement in the platform, and a base, can be assembled into operative devices for measuring balance. A photographic image of an exemplary prototype is shown in FIGS. 4 and 5. FIG. 4 demonstrates the operative prototype including a base, platform and sides as described previously. FIG. 5 demonstrates the operative prototype with the top platform and a housing panel removed to reveal a pair of actuators which induce movements within the platform. In the device two actuators are shown to be oriented in different directions as described in embodiments of the disclosure.

As described herein, a platform is a substantially flat surface that can support the weight of a subject without deforming. The platform can be made of many materials including composites, plastics, metals, or combinations thereof. Additionally the platform shape and surface are not limited to any specific values. In one example, the platform shape can be a polygon, such as a rectangle, square, or other polygon, or a circle or an oval of any other shape. The platform can have a surface area of various sizes. The platform can be large enough to accommodate a person standing with feet at shoulder width, a person standing with one foot in front of the other, or a person standing on one foot.

An embodiment of the disclosure can include adjusting the platform by varying the platform angle, platform surface or both. Such adjustments could be used to increase or decrease the challenge to the subject positioned thereon. For instance, the platform angle can be varied so that the platform surface is no longer normal to gravity. To adjust the platform surface, the device can further include a platform cover between the platform and the subject. The platform cover may be used to modify the platform surface by providing incline, cushion, or friction. In one example, a foam pad can be used as the platform cover. In another example, a sheet of sandpaper can be used as the platform cover. In an additional example, a 10° incline can be used as the platform cover. Further these platform covers can be used individually or in combination.

As described herein, the mechanism includes one or more mechanical components to move or alter the position of the platform. Any component that one of ordinary skill would use to achieve movement in a platform can be applied in the device. For example a pneumatic actuator could provide the component of the device. Alternatively, one or more ball bearing sliders could provide the component of the device. Further, many different types of actuators can provide the component of the device, according to what one of ordinary skill in the art would apply to provide movement to the platform. The following list is provided for illustrative purposes and should not be considered as limiting the types of different actuators one could provide as the component of the device: hydraulic actuator, pneumatic actuator, electric actuator, thermal actuator, and mechanical actuator. Combinations of components can also be used, such as a device including one or more actuators and one or more ball bearing sliders.

Further, it is contemplated that alternative devices may include components for inducing movement in a z-direction perpendicular to the horizontal plane. For example, a leaver, hydraulic actuator or combination positioned underneath platform can provide a vertical platform movement. The leaver, hydraulic actuator or combination thereof can be positioned provide distributed force to the bottom of the platform so that the vertical platform movement does not alter the platform angle. Alternatively the leaver, hydraulic actuator or combination thereof can provide localized force to the bottom of the platform to induce platform movements that include tilt, such that one or more of the platform sides increases in height, decreases in height, or both.

Devices for measuring balance may also include a base, support rails, or both to support the platform during device operation. An example of these devices includes a base connected to the mechanism. Preferably the base covers a larger area than the platform. The base may also be detachable from the mechanism to enhance device portability.

Devices for measuring balance in a subject may include at least one sensor to measure a data set comprising the subject's response to the platform movement. These sensors can include any sensor configured to transmit a signal proportional to the applied force, such as a load cell.

Devices for measuring balance in a subject may further include a storage location to contain the data set measured by the sensor. The storage location provides a digital or analog repository that records the data set measured by the sensor. Several non-limiting examples of a storage location can include a server, a computer, or a paper file. Additionally these storage locations can be either remote or local to the device. When the storage location includes a remote location, the device can also include a wireless transmitter to directly send the data set to the remote storage location, such as a cloud server.

Devices can further include different combinations of the number, position, and type of sensor. In some embodiments, the platform includes at least 2 sensors. In other embodiments, the platform includes at least 4 sensors. The platform can include 1 sensor, 2 sensors, 3 sensors, 4 sensors, 5 sensors, 6 sensors or more than 6 sensors. The sensor or sensors may be integrated in the platform, may be atop the platform or may be positioned at the perimeter of the platform. At least one sensor may be positioned near the center of the platform. While preferred sensors include load cells, any sensor capable of producing a dynamic force measurement may be adapted for use in embodiments of the disclosure.

Devices may further include at least one mode to process the data set measured by the sensor. These modes can be applied in combination or individually. For example, a Mode I could provide a microprocessor and a display for real-time analysis of the data set which may be visualized by the subject on the display. A Mode II could provide a wireless transmitter for transmitting the data set to a remote server or cloud. The data set may go through pre-processing either before, or concurrent with a mode to process the data set. Methods for pre-processing can include but are not limited to: adaptive filtering, thresholding, and linear filtering.

Elements of the disclosure can include the platform movement that comprises a platform displacement, a platform velocity, and a platform acceleration. The platform displacement provides a change in location of the platform from a starting position having a first coordinate location (x0, y0) to a final position having a second coordinate location (x1, y1). The platform displacement covers a distance (d) calculated using equation 1.

d=\sqrt{(x1−x0)²+(y1−y0)²} Eq. 1

The platform velocity and platform acceleration can be described according to ordinary Newtonian mechanics. As such velocity is used to describe a time-dependent change in platform position and acceleration is used to describe a time-dependent change in platform velocity. Both the platform velocity and the platform acceleration are vectors which can be defined by a magnitude and a direction.

Devices for measuring balance may include an interface in communication with the device. This interface provides control signals to adjust or program the platform movement. In an exemplary device platform velocity and platform acceleration can be dynamically controlled through the interface, such that platform movement could be adjusted in real time. Alternatively, platform velocity and platform acceleration can be random. In another alternative device the platform movement can be dynamic, preset, random or a combination thereof.

As described herein, the subject positioned atop the platform experiences a force due to the acceleration component of the platform movement, which can be measured as the subject's response to the platform movement. Devices provided by the disclosure may also include closed loop control so that the force due to acceleration is independent of the weight of the subject. In a non-limiting exemplary device, a potentiometer can be included to track platform location and provide motor feedback to achieve the desired kinematics. For example, the potentiometer can sense the platform is moving slower due to increased load on the platform and adjust power output in real time. Further, a subject's position or stance atop the platform can change the dynamic load which could be sensed by the potentiometer, or similar closed loop control mechanism, to ensure desired kinematics.

Devices may also include an adapter configured to connect a power source. Power sources may provide either AC or DC current and can include combinations or individual sources. In one embodiment, an adapter is configured to connect a battery to provide DC current. In another embodiment, a first adapter is configured to connect a battery to provide DC current and second adapter is configured to connect a wall outlet to provide AC current.

A non-limiting exemplary device of the disclosure can include a substantially rectangular platform containing 1 load cell positioned proximate the platform perimeter. The device includes a mechanism connected to the platform which induces a platform movement, and the load cell measures a data set including the subject's response to the platform movement.

Another non-limiting exemplary device of the disclosure can include a substantially rectangular platform containing two load cells positioned proximate the platform perimeter. The device includes a mechanism connected to the platform which induces a platform movement, and the load cells measure a data set that includes the subject's response to the platform movement. The platform movement by the mechanism can include an x-direction and y-direction, independent of one another and optionally oriented perpendicular to one another.

Another non-limiting exemplary device of the disclosure can include a substantially rectangular platform containing four load cells positioned proximate the platform perimeter at each edge of the platform. The device includes a mechanism connected to the platform which induces a platform movement. The platform movement by the mechanism can include an x-direction and y-direction, independent of one another and optionally oriented perpendicular to one another. The load cells measure a data set that includes the subject's response to the platform movement.

Another non-limiting exemplary device of the disclosure can include a substantially rectangular platform containing four load cells positioned proximate the platform perimeter at each corner of the platform. The device includes a mechanism connected to the platform which induces a platform movement. The platform movement by the mechanism can include an x-direction displacement, a y-direction displacement, a platform velocity, and a platform acceleration. The device also includes an interface for controlling the platform movement. The load cells measure a data set that includes the subject's response to the platform movement.

The disclosure also includes methods to measure balance in a subject. The methods for measuring balance can include positioning the subject on a platform, inducing a series of platform movements, and measuring a data set comprising the subject's response to each platform movement using at least one sensor incorporated in or on the platform.

As described herein, positioning the subject on the platform can include at least one of the following stances: one leg stance, double leg wide stance, double leg narrow stance and tandem stances. These stances are not limited in practice and a series of platform movements could include multiple stances such as double leg narrow stance and one leg stance.

As described herein, inducing a platform movement can include one or more components oriented in various directions. An exemplary method may include two components: a first component that induces an x-direction platform movement and a second component that induces a y-direction platform movement. The first component and second component may act and operate independent of one another such that a platform movement could comprise an x-direction platform movement, a y-direction platform movement, or a combination of the two. Additionally these movements may occur concurrently or in series. In one possible scenario, a method for assessing balance induces an x-direction platform movement. In a second scenario, a method for assessing balance induces an x-direction platform movement and a y-direction platform movement concurrently. In a third scenario, a method for assessing balance induces an x-direction platform movement and a y-direction platform movement sequentially.

As described herein, the x-direction platform movement is directed to a displacement along an x-axis of motion and the y-direction platform movement is directed to a displacement along an y-axis of motion. Both the x-direction platform movement and the y-direction platform movement may produce both positive and negative displacement along each respective axis. Thus an x-direction platform movement could include a positive displacement from (0, 0) to (5, 0), a negative displacement from (0, 0) to (−5, 0), or a combination of a positive displacement and a negative displacement from (0, 0) to (5, 0) to (0, 0). Similarly a y-direction platform movement could include a positive displacement from (0, 0) to (0, 5), a negative displacement from (0, 0) to (0, −5), or a combination of a positive displacement and a negative displacement from (0, 0) to (0, 5) to (0, 0).

Additionally, methods of the disclosure are not dependent on the device used. Unless specifically noted, methods of use can be applied to any device capable of performing the described steps.

Devices can further include a reset after the platform movement. As described herein, the reset after the platform movement includes a reset time and a neutral platform position. The reset may be used to control the difficulty by reducing learning and ceiling effects when the device is used to induce multiple platform movements. The reset time and the neutral platform position can be predefined or random depending on the intended use. The reset time can include a time period where the platform, subject, and device are motionless.

Methods for measuring balance include a platform, or other flat surface that can support the weight of a subject without deforming and can move. The platform can be made of many materials including composites, plastics, metals or combinations thereof. Methods of use are not limited to any specific values of platform shape or surface. In one example, the platform shape can be a polygon, such as a rectangle, square, or other polygon, or a circle or an oval of any other shape. The platform can have a surface area of various sizes. The platform can be large enough to accommodate a person standing with feet at shoulder width, a person standing with one foot in front of the other, or a person standing on one foot.

Methods measuring a data set may include different combinations of the number, position, and type of sensor. In some methods, the platform includes at least 2 sensors. In other embodiments, the platform includes at least 4 sensors. The platform can include 1 sensor, 2 sensors, 3 sensors, 4 sensors, 5 sensors, 6 sensors or more than 6 sensors. The sensor or sensors may be integrated in the platform, may be atop the platform or may be positioned at the perimeter of the platform. At least one sensor may be positioned near the center of the platform. While preferred sensors include load cells, any sensor capable of producing a dynamic force measurement may be adapted for use in embodiments of the disclosure.

Also disclosed herein are methods for assessing or training balance in a subject, which may include the following steps: positioning the subject on a platform, which includes at least one sensor and is configured to move along a horizontal plane; inducing a series of platform movements in the horizontal plane; measuring a data set comprising the subject's response to each platform movement using at least one sensor incorporated in or on the platform; storing the data set in a storage location; and reporting a balance assessment for the subject.

Optionally, the storage location can provide a reference tag associated with the data set comprising a subject ID and a session ID. Both the subject ID and session ID provide an organizational tool to compare subject balance over time, between different subjects, or both. The subject ID can be associated with a unique subject profile, containing information which can include: subject name, subject height, subject weight, and/or subject medical history. The session ID can be associated with a time stamp and can include: date and/or number of platform movements.

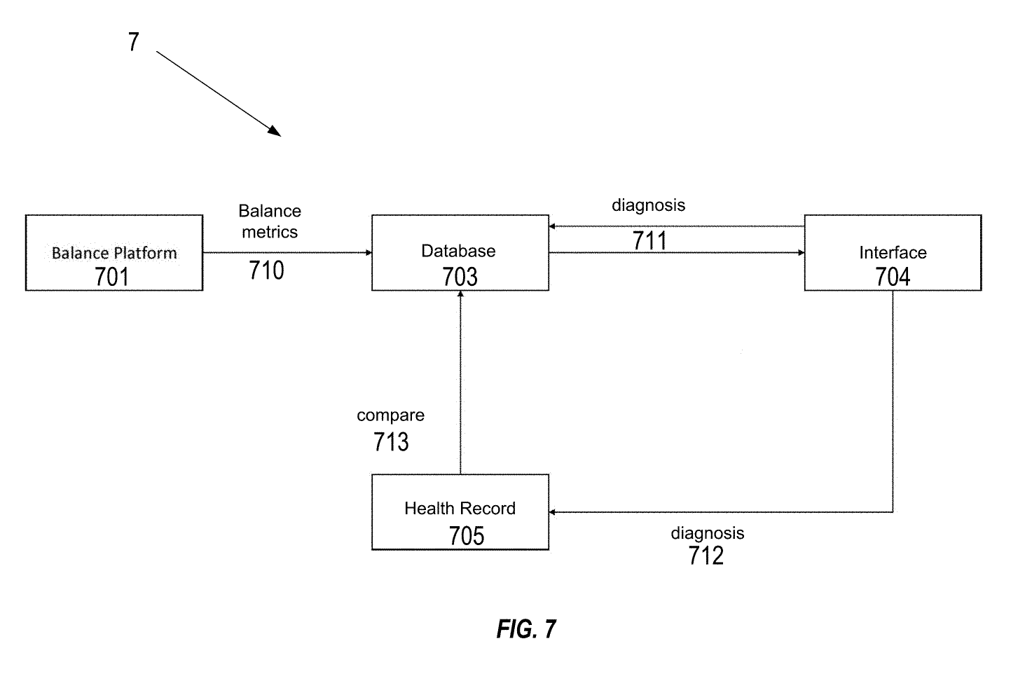

FIG. 7 displays a non-limiting, exemplary method of assessing balance 7 in accordance with embodiments of the disclosure. A balance platform 701 is used to extract balance metrics 710 from a subject which are then transmitted to a database 703. The database is in communication with an interface 704 and a health record 705. The database 703 provides a location to compare 713 balance metrics 710 with the health record 705. Further, the interface allows a doctor, physical therapist, or other health professional to update the database by providing a diagnosis 711, update the health record with a diagnosis 712, or update both diagnosis 711 and diagnosis 712.

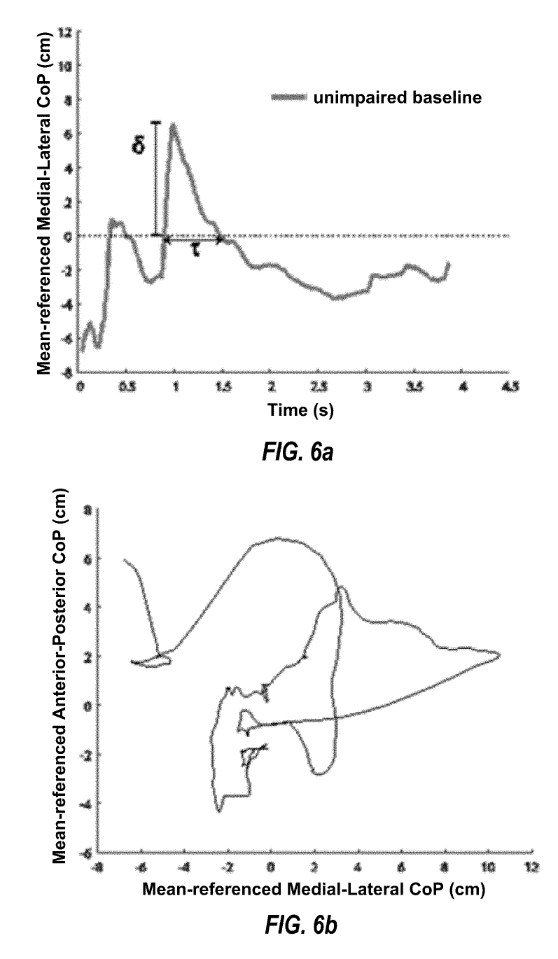

Methods for data processing may include relating the applied force to calculate a center of pressure (COP) measurement. Additionally measuring the applied force over the platform can produce positional center of pressure measurements such as medial-lateral COP and anterior-posterior COP. This data can be further processed to determine characteristic parameters which can be related to the balance of the subject. In one exemplary method, the sensors measure a data set which is processed to generate a COP as a function of time. The COP may be pre-processed by normalizing the measurements, such as by referencing the measurements to a mean or average value, or referencing the measurements to an initial value. Characteristic parameters can include the peak excursion (δ), defined as the maximum COP and the recovery time (τ), and defined as the time it takes for a baseline COP to adjust to 6 and then return to the baseline COP. In another scenario, the time dependent response in pressure or force can be combined with the platform kinematics. FIGS. 6a and 6b display data sets measured or calculated in accordance with an exemplary method of the disclosure. Additionally characteristic parameters such as peak excursion and recovery time are indicated.

Optionally, the data set may be pre-processed using at least one of the following tools: adaptive filtering, thresholding, or linear filtering.

As described herein, balance metrics may refer to either a single data set, a processed data set, or characteristic parameters derived from the data set or processed data set. These balance metrics may be hosted in an external database accessible through the internet, or stored locally.

An exemplary method for assessing or training balance may include tracking a subject's balance over the course of treatment or physical therapy. During this period, at least two data sets comprising the subject's response to a series of platform movements would be measured and stored. By comparing the data sets over the course of treatment or physical therapy, a change in balance characteristic parameters can be used to objectively track patient recovery.

In one scenario, the method for assessing balance can include inducing at least 5-20 platform movements. In a second scenario, the method for assessing balance can include inducing at least 7 platform movements. In a third scenario, the method for assessing balance can include at least 10 platform movements. Greater numbers of platform movements could be used increase challenge of the balance assessment, thus alternative scenarios may include up to 25 platform movements or up to 50 platform movements.

In another scenario, the method for assessing balance can further include positioning the subject in one leg stance for at least 2-10 platform movements.

Methods for assessing or training balance may further include a diagnosis using subject characteristics including the data set, medical history, and height or weight measurements. The diagnosis can be selected from one or more of the following group: concussion, inebriation, chemical impairment, and central nervous system depression.

Embodiments of the disclosure can further include tracking the subject balance by comparing at least two data sets. The at least two data are directed to the same subject ID but preferably include different session IDs. To track subject balance, the change in one or more balance metrics over time can be used. To determine the change in balance metrics, at least two data sets are needed. The two data sets are associated with the same subject ID, indicating that were both measured for the same subject. The two data sets are also associated with different session IDs, indicating that the data sets were measured at different times or on different dates. Tracking the subject balance includes at least a step of associating a data set with a unique subject, and a step of associating a data set with a unique time stamp. The combination of both these steps produces a record of balance metrics that can be used to evaluate and/or assess the balance of a subject over time, such as to evaluate balance improvement while undergoing physical therapy.

A non-limiting exemplary method of the disclosure can include positioning a subject on a substantially rectangular platform containing four load cells positioned near the corners of the platform, inducing a series of platform movements, measuring a data set and dynamically processing the data set by interpolating between the sensors to calculate the position of the center of pressure over time.

### Examples

An exemplary device as described herein was used to measure balance in a subject as shown in FIGS. 6a and 6b. The subject was positioned on the platform in a specified stance with arms folded across their chest while the device induced a series of random platform movements in the horizontal plane. During the platform movements the subject was instructed to maintain balance without changing their stance or moving their arms. The series of platform movements occurred over a period of about 1 to about 2 minutes which included about 15 to about 30 platform movements. The device processed sensor data to calculate mean-referenced medial-lateral CoP and mean-referenced anterior-posterior CoP as shown in FIG. 6b. The mean-referenced medial-lateral CoP was further processed to determine the characteristic parameters of peak excursion and recovery time as shown in FIG. 6a.

We claim:

1. A device for measuring balance in a subject positioned thereon comprising:
i. a platform;
ii. a sensor; and
iii. a mechanism connected to the platform; wherein the mechanism induces a platform movement along a horizontal plane and the sensor measures a data set comprising the subject's response to the platform movement.

2. The device of claim 1 wherein the platform movement comprises an x-direction and a y-direction, independent of one another and oriented perpendicular to one another in the horizontal plane.

3. The device of claim 1 wherein the platform movement comprises: a platform displacement, a platform velocity, and a platform acceleration.

4. The device of claim 1 further comprising a base, wherein the base is connected to the mechanism at a position different from the platform.

5. The device of claim 1 wherein the sensor is a load sensor or a pressure sensor for measuring force of the subject positioned on the platform.

6. The device of claim 1 comprising at least 2 sensors.

7. The device of claim 1 wherein the mechanism comprises at least one actuator.

8. The device of claim 1 wherein the platform is substantially rectangular and comprises 4 load cells positioned proximate to the platform perimeter.

9. The device of claim 8 wherein the platform movement comprises an x-direction and a y-direction, independent of one another and oriented perpendicular to one another in the horizontal plane.

10. The device of claim 9 wherein the platform movement comprises: an x-direction displacement, a y-direction displacement, a platform velocity, and a platform acceleration.

11. A method for measuring balance in a subject comprising:
i. positioning the subject on a platform, wherein the platform comprises at least one sensor and is configured to move along a horizontal plane;
ii. inducing a series of platform movements in the horizontal plane; and
iii. measuring a data set comprising the subject's response to each platform movement using at least one sensor incorporated in or on the platform.

12. The method of claim 11 further comprising a platform reset after each platform movement, wherein the platform reset comprises a reset time and a neutral platform position.

13. The method of claim 12 further comprising saving the data set in a storage location, wherein the storage location provides a reference tag comprising a subject ID and a session ID.

14. The method of claim 11 wherein the sensor is a load sensor or a pressure sensor for measuring force of the subject positioned on the platform.

15. The method of claim 11 wherein the platform comprises at least 2 sensors.

16. The method of claim 11 wherein the mechanism comprises at least one actuator.

17. The method of claim 11 wherein inducing a series of platform movements in the horizontal plane comprises providing a power source to transfer energy to a mechanism connected to the platform.

18. The method of claim 11 wherein each platform movement comprises an x-direction and a y-direction, independent of one another and oriented perpendicular to one another in the horizontal plane.

19. The method of claim 11 wherein each platform movement comprises an x-direction displacement, a y-direction displacement, a platform velocity, and a platform acceleration.

20. The method of claim 19 wherein the series of platform movements comprises at least 8 platform movements.
